(19) United States
(12) Patent Application Publication
(10) Pub. No.: US 2025/0239352 A1
(43) Pub. Date: Jul. 24, 2025

(54) CHATBOT FOR PROMOTING EXERCISE HABIT FORMATION OF PATIENT WITH CHRONIC DISEASES

(71) Applicant: Peking Union Medical College, Beijing (CN)

(72) Inventors: Meihua PIAO, Beijing (CN); Haoming MA, Beijing (CN); Hongzhen CUI, Beijing (CN); Runyuan PEI, Beijing (CN); You ZHOU, Beijing (CN); Aoqi WANG, Beijing (CN); Xingyi TANG, Beijing (CN); Sijia LI, Beijing (CN)

(21) Appl. No.: 18/627,292

(22) Filed: Apr. 4, 2024

(30) Foreign Application Priority Data
Jan. 24, 2024 (CN) 202410100790.4

(51) Int. Cl. G16H 20/30; G06F 21/31; G06F 21/62; G06F 40/20; G16H 10/60; G16H 50/20

(52) U.S. Cl. CPC G16H 20/30; G06F 21/31; G06F 21/6254; G06F 40/20; G16H 10/60; G16H 50/20

(57) ABSTRACT

The present disclosure discloses a chatbot for promoting exercise habit formation of a patient with chronic diseases, including: a user information and health record module configured to store a patient's personal information, health status and medical history; a personalized modeling and recommendation system module configured to establish a personalized exercise plan recommendation system based on user information and a health record; a neuro linguistic programming (NLP) module configured to process a natural language input of a user and provide a dialogue; a real-time feedback system module configured to provide a real-time exercise feedback, including exercise data and suggestions; an exercise data acquisition and analysis module configured to obtain the patient's exercise data from an exercise tracking device or application for analysis and interpretation; a multi-channel integration module configured to enable the chatbot to run on different platforms; a social interaction module configured to provide a social interaction function.

### CROSS REFERENCE TO RELATED APPLICATION

This patent application claims the benefit of and priority to Chinese Patent Application No. 202410100790.4, filed with the Chinese Patent Office on Jan. 24, 2024, which is hereby incorporated by reference herein in its entirety.

### TECHNICAL FIELD

The present disclosure relates to the technical field of artificial intelligence, and in particular, to a chatbot for promoting exercise habit formation of a patient with chronic diseases.

### BACKGROUND

Formation of exercise habits of patients with chronic diseases is of great significance for improving a health status, controlling diseases, and improving quality of life. The patients with chronic diseases can be helped to form good exercise habits. For example, before starting any new exercise plan, the patients should consult a doctor, who can evaluate health statuses of the patients and provide customized exercise suggestions to ensure that an exercise plan is coordinated with a treatment plan. There is also a need to set reasonable and feasible exercise goals, gradually increase the intensity and time of exercise, and take into account the patient's physical condition, medical history and the doctor's advice to ensure that the goals will not have a negative impact on health. Therefore, for the formation of exercise habits of the patients with chronic diseases, there is a need to comprehensively consider individual differences and medical conditions of the patients and formulate a reasonable exercise plan, which is the key to ensure that the patients can form exercise habits safely and effectively.

### SUMMARY

To solve the above problems, the present disclosure provides a chatbot for promoting exercise habit formation of a patient with chronic diseases.

To achieve the above objectives, the technical solution adopted by the present disclosure is as follows:

A chatbot for promoting exercise habit formation of a patient with chronic diseases includes:
a user information and health record module configured to store a patient's personal information, a health status and medical history and connected to a personalized modeling and recommendation system module;
the personalized modeling and recommendation system module configured to establish a personalized exercise plan recommendation system based on user information and a health record and connected to the user information and health record module and a neuro linguistic programming (NLP) module;
the NLP module configured to process a natural language input of a user and provide a dialogue and connected to the personalized modeling and recommendation system module, the user information and health record module, and a real-time feedback system module;
the real-time feedback system module configured to provide a real-time exercise feedback, including exercise data and suggestions, and connected to the personalized modeling and recommendation system module, an exercise data acquisition and analysis module, and the NLP module;
the exercise data acquisition and analysis module configured to obtain the patient's exercise data from an exercise tracking device or application for analysis and interpretation and connected to the real-time feedback system module and the personalized modeling and recommendation system module;
a multi-channel integration module configured to enable the chatbot to run on different platforms and connected to the personalized modeling and recommendation system module, the user information and health record module, and the NLP module;
a social interaction module configured to provide a social interaction function and connected to the personalized modeling and recommendation system module and the NLP module; and
a security and privacy module configured to ensure that the patient's health data is securely stored and processed, and connected to the user information and health record module.

Further, the user information and health record module is configured to:
use a database to store the patient's personal information and health record, including name, age, gender, physical condition, past medical history, and medication;
implement data encryption for storage of personal health information;
introduce a user authentication mechanism to ensure that only legally authorized personnel access and modify the patient's information;
provide an application programming interface (API) so that other modules access the patient's information;
update the database when the patient updates the personal information or has a new medical record; and
provide a user interface to enable the patient to conveniently view and edit the personal information.

Further, the personalized modeling and recommendation system module is configured to:
establish a personalized model by using a data analysis technology based on the patient's personal information and health record data, to identify the patient's exercise preference, physical condition and response mode;
develop a rule engine to make an exercise plan for the patient based on medical knowledge and health guidelines;
adjust the exercise plan when the patient's health status changes or a new medical record is added;
introduce a feedback loop to continuously adjust the model by monitoring the patient's exercise feedback and progress;
ensure, by means of an API, that the personalized modeling and recommendation system module accesses and updates data of the user information and health record module; and
enable, by the NLP module, the personalized exercise plan recommendation system to understand a natural language input of the patient, and adjust an output of the personalized model based on the patient's questions and needs.

Further, the NLP module is configured to:
convert the user's oral input into text;
use a natural language processing technology to analyze and understand the user's text input to extract a user intention and key information;
consider integrating emotional analysis to understand the user's emotional state;
extract a key entity in the user input by using an entity recognition technology;
introduce dialogue management to keep a context of the dialogue and ensure coherence and fluency;
determine the user's specific intention through intention recognition; and
ensure, by means of an API, that the NLP module is connected to the personalized modeling and recommendation system module, the user information and health record module, and the real-time feedback system module.

Further, the real-time feedback system module is configured to:
connect to the exercise data acquisition and analysis module to receive the real-time exercise data;
perform real-time processing and analysis on the received exercise data; and use a fast and effective algorithm including a real-time data stream processing technology, to ensure timeliness and accuracy;
generate a personalized exercise feedback based on an analysis result;
ensure real-time performance of feedback information, to enable the patient to know the patient's own exercise status and obtain suggestions in time, and achieve personalized adjustment of a real-time feedback with the personalized modeling and recommendation system module;
connect to the NLP module, receive the user's natural language input, and adjust feedback content in time; and
ensure, by means of an API, that the real-time feedback system module is connected to the personalized modeling and recommendation system module.

Further, the exercise data acquisition and analysis module is configured to:
access the exercise tracking device or application;
communicate with the exercise tracking device or application by means of an API to obtain the real-time exercise data;
clean and preprocess collected original data to deal with an abnormal value and a missing value;
use an efficient data acquisition algorithm to ensure real-time performance of the data;
use a precise data analysis algorithm to ensure accuracy of the exercise data;
use a caching and streaming technology to achieve real-time processing of a large data volume;
use a machine learning technology to analyze the exercise data, including exercise pattern recognition, exercise habit analysis, and exercise effect evaluation, to obtain deeper exercise information;
generate a readable analysis report based on an analysis result, including the patient's exercise trend and health status evaluation; and
ensure, by means of an API, that the exercise data acquisition and analysis module is connected to the real-time feedback system module and the personalized modeling and recommendation system module.

Further, the multi-channel integration module is configured to:
design an API, so that a function of the chatbot is invoked by means of the interface;
use a responsive design principle, so that an interface of the chatbot adapts to screen sizes and resolution of different terminals;
develop a mobile application platform and make full use of hardware functions of a mobile device;
create a Web-based user interface, so that the user accesses the chatbot via a browser;
use an API authentication and authorization mechanism to ensure secure and reliable data transmission on different platforms;
achieve adaptive design, so that the chatbot automatically adjusts a layout and functions on different platforms based on a user device and environment; and
ensure, by means of the API, that the multi-channel integration module is connected to the user information and health record module and the personalized modeling and recommendation system module.

Further, the social interaction module is configured to:
provide a user registration function to allow the patient to create a personal account and fill in personal data;
achieve a user search and matching function, so that the patient finds and connects to other users with similar interest or the same health goal;
provide a real-time chat function, so that patients interact directly;
allow the patient to share exercise achievements and progress;
allow the user to create an exercise group for a wider range of social interaction;
allow the user to know group activities and new chat information in time by using a push notification function;
provide a channel for the user to provide a feedback and suggestions to continuously optimize functions of the social interaction module; and
ensure, by means of an API, that the social interaction module is connected to the personalized modeling and recommendation system module.

Further, the security and privacy module is configured to:
encrypt the patient's health data to ensure security during storage and transmission;
implement an access control mechanism to ensure that only authorized personnel access the patient's health data;
anonymize the health data to reduce an identification risk of the individual patient;
implement a security audit and monitoring mechanism to track a security event and an access record in the chatbot;
implement fine-grained user permission management in the chatbot to ensure that each user only accesses data and functions required by the user's job responsibilities;
regularly update security of the chatbot, fix known vulnerabilities in time, implement a vulnerability management mechanism, and regularly perform security assessment and penetration testing; and
ensure, by means of an API, that the security and privacy module is connected to the user information and health record module.

Compared with the prior art, the present disclosure has the following technical progress.

By a personalized modeling and recommendation system, the chatbot can generate customized exercise suggestions based on the patient's personal information and health record, which go beyond conventional universal suggestions, meet the patient's personalized needs more effectively, and improve the effect of forming exercise habits. A neuro linguistic programming (NLP) module of the chatbot can understand the patient's natural language input and achieve a more natural and intelligent dialogue experience, which enables the patient to communicate with the chatbot more easily, provides more accurate information, and helps the patient to more actively participate in the cultivation of exercise habits.

By introducing a real-time feedback system, the chatbot can obtain the patient's exercise data in time and generate a real-time personalized feedback, which helps the patient to keep exercising more actively, and provides timely adjustment suggestions to make an exercise plan more suitable for the patient's actual situation.

By integrating an advanced exercise tracking device or application, the chatbot can obtain real-time exercise data of the patient. An exercise data acquisition and analysis module uses an efficient algorithm to ensure real-time performance and accuracy of data, which helps to provide richer and more reliable data support for personalized modeling and improve accuracy of exercise suggestions. By a multi-channel integration module, the chatbot can run on different platforms, including a mobile application and a web page, which provides the patient with more flexible choices, and enables the patient to interact with the chatbot anytime and anywhere, thereby increasing user engagement. By introduction of a social interaction module, patients can share experiences and establish communities, which promotes interaction and support between the patients, provides additional incentives, and helps to maintain and strengthen exercise habits. By a security and privacy module, security and privacy of the patient's health data are ensured, and an advanced encryption technology and access control measures are used to ensure that the patient's personal health information is fully protected.

To sum up, the present disclosure provides more intelligent, more personalized and more comprehensive exercise suggestions and support for the patient by comprehensively applying personalized modeling, natural language processing, real-time feedback, exercise data acquisition and analysis, and other technologies, thus more effectively promoting the formation of exercise habits of the patient with chronic diseases.

### BRIEF DESCRIPTION OF THE DRAWINGS

Accompanying drawings are intended to provide a further understanding of the present disclosure, and constitute a part of the description. With the embodiments of the present disclosure, the accompanying drawings are intended to explain the present disclosure, and do not constitute a limitation on the present disclosure.

In the FIGURE,

FIG. 1 is a structural diagram of a system according to the present disclosure.

### DETAILED DESCRIPTION OF THE EMBODIMENTS

The following several specific embodiments can be combined with each other, and the same or similar concepts or processes may not be described in detail again in some embodiments. The embodiments of the present disclosure will be described below with reference to the accompanying drawings.

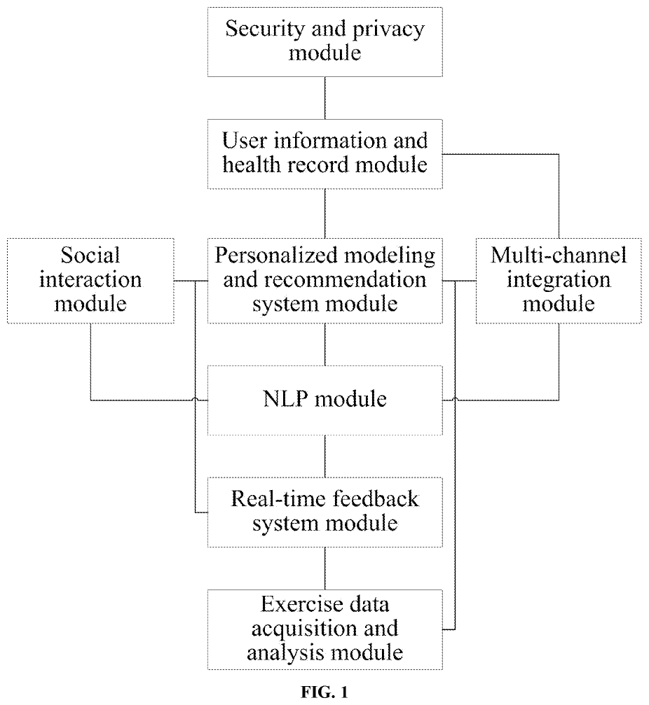

As shown in FIG. 1, the present disclosure discloses a chatbot for promoting exercise habit formation of a patient with chronic diseases, including:
a user information and health record module configured to store a patient's personal information, a health status and medical history, and the like, and connected to a personalized modeling and recommendation system module;
the personalized modeling and recommendation system module configured to establish a personalized exercise plan recommendation system based on user information and a health record and connected to the user information and health record module and an NLP module;
the NLP module (natural language processing) configured to process a natural language input of a user, understand user needs, and provide a natural and smooth dialogue experience, and connected to the personalized modeling and recommendation system module, the user information and health record module, and a real-time feedback system module;
the real-time feedback system module configured to provide a real-time exercise feedback, including exercise data, suggestions, and the like, and connected to the personalized modeling and recommendation system module, an exercise data acquisition and analysis module, and the NLP module;
the exercise data acquisition and analysis module configured to obtain the patient's exercise data from an exercise tracking device or application for analysis and interpretation, connected to the real-time feedback system module and the personalized modeling and recommendation system module, and
solving problems by using an efficient data acquisition and analysis algorithm to ensure real-time performance and accuracy;
a multi-channel integration module configured to enable the chatbot to run on different platforms, such as a mobile application and a web page, and connected to the personalized modeling and recommendation system module, the user information and health record module, and the NLP module;
a social interaction module configured to provide a social interaction function to allow the patient to share experiences and establish a community, and connected to the personalized modeling and recommendation system module and the NLP module;
a security and privacy module configured to ensure that the patient's health data is securely stored and processed, and connected to the user information and health record module.

A collaboration process between modules is as follows:

The user information and health record module provides basic information of a user;
the personalized modeling and recommendation system module makes a personalized exercise plan based on the user information;
the user communicates in natural language by means of the NLP module, and asks questions or obtains suggestions;
the exercise data acquisition and analysis module obtains a patient's exercise data by integrating an exercise tracking device;
the real-time feedback system module provides a real-time exercise feedback based on an analysis result and suggests appropriate exercise adjustment;
the social interaction module encourages the user to share exercise experiences and establish mutual assistance in a community;
the multi-channel integration module ensures that the user can seamlessly use the chatbot on different platforms; and
the security and privacy module is responsible for protecting health data security of the user.

Specifically, the user information and health record module is implemented in the following way:

Storage by using a database: The database is used to store the patient's personal information and health record, including but not limited to name, age, gender, physical condition, past medical history, medication, and the like. A relational database or a NoSQL database is used to store the information for quick retrieval and update.

Data encryption: Implementing data encryption is essential for storage of personal health information. A powerful encryption algorithm is used to protect the patient's privacy and ensure that sensitive information is not accessed without authorization.

User authentication: A user authentication mechanism is introduced to ensure that only legally authorized personnel can access and modify the patient's information. This can be achieved by means of a user name and a password, two-factor authentication, or the like.

Interface and standard: A standard application programming interface (API) is provided, so that other modules can access the patient's information. A health information exchange standard, such as HealthLevelSeven (HL7), is adopted, to ensure compatibility of data interaction with other medical systems.

Real-time synchronization: Real-time synchronization of the user information and health record module with other related modules is ensured. When the patient updates the personal information or has a new medical record, the database is updated in time to ensure that other modules obtain the latest information.

User interface (UI): A user-friendly interface is provided to enable the patient to conveniently view and edit the personal information. This may be a special patient-side application, or may be presented in the form of a web page or a mobile application.

Connection to the personalized modeling and recommendation system module: By means of a clearly-defined API, effective connection between the user information and health record module and the personalized modeling and recommendation system module is ensured. A personalized modeling and recommendation system needs real-time access to the patient's information to adjust exercise suggestions based on a latest health status.

By means of the above implementation, the user information and health record module can effectively store, protect and provide the patient's personal information, and cooperate with other modules to provide health record-based personalized services for the chatbot.

Specifically, the personalized modeling and recommendation system module is implemented as follows:

Data analysis and modeling: A personalized model is established by using a data analysis technology based on the patient's personal information and health record data. This may include a machine learning algorithm, such as a decision tree, a neural network, and the like, to identify the patient's exercise preference, physical condition, response mode, and the like.

Rule engine: A rule engine is developed to make an exercise plan for the patient based on medical knowledge and health guidelines. This may be a system based on expert rules, or a system that combines machine learning and rules, to improve accuracy of recommendation.

Real-time update: The model needs to be updated in real time to adapt to changes of the patient. For example, when the patient's health status changes or a new medical record is added, the model should be able to quickly adjust the exercise plan.

Feedback loop: The feedback loop is introduced to continuously adjust the model by monitoring the patient's exercise feedback and progress. This can be achieved by means of the patient's real-time data, exercise report, health questionnaire, and the like.

Connection to the user information and health record module: By means of a clearly-defined API, it is ensured that the personalized modeling and recommendation system module can access and update data of the user information and health record module. This helps to establish accuracy of the personalized model.

Integration of the NLP module: By the NLP module, the system can understand a natural language input of the patient, and adjust an output of the personalized model based on the patient's questions and needs. This can achieve a more intelligent and natural dialogue experience.

Security considerations: As the patient's personal health information is involved, it is ensured that the established personalized model complies with relevant privacy and security standards. Anonymization, encryption and other means are used to protect the privacy of the patient.

By means of the above implementation, the personalized modeling and recommendation system module can make full use of the patient's personal information and health record to establish an accurate personalized exercise plan recommendation system updated in real time, and is connected to other key modules to provide customized exercise suggestions for the patient.

Specifically, the NLP module (natural language processing) is implemented as follows:

Speech recognition technology: An advanced speech recognition technology is used to convert the user's oral input into text. This can be achieved by integrating an existing speech recognition API or using a deep learning model.

Text analysis and understanding: A natural language processing technology is used to analyze and understand the user's text input. This includes lexical analysis, grammatical analysis, semantic analysis, and the like, to extract a user intention and key information.

Emotional analysis: Integrating emotional analysis is considered to understand the user's emotional state. This helps the chatbot to better adapt to the user's emotions and provide a more appropriate response.

Entity recognition: A key entity in the user input, such as a time, a place, and an exercise type, is extracted by using an entity recognition technology. This helps to understand the user's needs and problems more accurately.

Dialogue management: A dialogue management system is introduced to keep a context of the dialogue and ensure coherence and fluency. The dialogue management system can use a rule-based method or a deep learning model to understand the context and generate natural responses.

Intention recognition: The user's specific intention, such as obtaining exercise suggestions and querying health information, is determined by using an intention recognition technology. This helps to connect user needs to corresponding modules.

Multilingual support: Considering that the user may communicate in different languages, it is ensured that the NLP module can support multilingual input, and can understand and respond.

Connection to other modules: By means of a clearly-defined API, it is ensured that the NLP module can be connected to the personalized modeling and recommendation system module, the user information and health record module, and the real-time feedback system module. This helps to convert the user's natural language input into a corresponding operation or information request.

By means of the above implementation, the NLP module can effectively process the natural language input of the user, understand user needs, achieve collaborative operation with other modules, and provide a natural and smooth dialogue experience.

Specifically, the real-time feedback system module is implemented as follows:

Exercise data receiving: The module is connected to the exercise data acquisition and analysis module to receive the real-time exercise data. This may include a number of steps, a heart rate, exercise duration, and other data, depending on the exercise tracking device or application used by the patient.

Data processing and analysis: Real-time processing and analysis are performed on the received exercise data. A fast and effective algorithm, for example, a real-time data stream processing technology, is used to ensure timeliness and accuracy.

Feedback generation: A personalized exercise feedback is generated based on an analysis result. This may include an encouraging language, exercise suggestions, goal achievement evaluation, and the like. A rule engine or a machine learning algorithm is used to ensure personalization and appropriateness of the feedback.

Real-time update: Real-time performance of feedback information is ensured, to enable the patient to know the patient's own exercise status and obtain suggestions in time, and achieve personalized adjustment of a real-time feedback with the personalized modeling and recommendation system module.

Communication coordination: The real-time feedback system module is connected to the NLP module to receive the user's natural language input, and adjust feedback content in time. This can be achieved by means of an event-driven mechanism to ensure that a response can be made to the user's feedback request in time.

UI presentation: The generated real-time feedback is presented to the patient via a user-friendly interface. This may be in the form of real-time notification of a mobile application, dynamic update on a web page, or the like.

Connection to the personalized modeling and recommendation system module: By means of a clearly-defined API, it is ensured that the real-time feedback system module can be connected to the personalized modeling and recommendation system module. This helps to combine the real-time exercise data with personalized modeling and provide more personalized and targeted suggestions.

By means of the above implementation, the real-time feedback system module can receive, process and generate a personalized exercise feedback in time, and is connected to other key modules to provide real-time and effective exercise suggestions and information for the patient.

Specifically, the exercise data acquisition and analysis module is implemented as follows:

Device access and compatibility: The module supports access of multiple exercise tracking devices or applications to ensure compatibility. This may include a smart bracelet, a smartwatch, a mobile phone application, and the like, to meet individual needs of the patient.

Data acquisition: The module communicates with the exercise tracking devices or applications by means of an API or another interface to obtain the real-time exercise data. It is ensured that the collected data includes a number of steps, a heart rate, exercise duration, and other key information.

Data cleaning and preprocessing: Collected original data is cleaned and preprocessed to deal with an abnormal value, a missing value, and the like. This helps to ensure accuracy and reliability of the analysis.

Real-time performance and accuracy: An efficient data acquisition algorithm is used to ensure real-time performance of the data. A precise data analysis algorithm is used to ensure accuracy of the exercise data. A caching and streaming technology and other technologies are used to achieve real-time processing of a large data volume.

Exercise data analysis: Statistics and the machine learning technology are used to analyze the exercise data. This may include exercise pattern recognition, exercise habit analysis, exercise effect evaluation, and the like, to obtain deeper exercise information.

Generation of an analysis report: A readable analysis report, including the patient's exercise trend and health status evaluation, and the like, is generated based on an analysis result. This helps the patient to better understand the patient's own exercise situation.

Connection to the real-time feedback system module: By means of a clearly-defined API, it is ensured that the exercise data acquisition and analysis module can be connected to the real-time feedback system module. This helps to use the analysis result to generate a real-time exercise feedback.

Connection to the personalized modeling and recommendation system module: By means of a clearly-defined API, it is ensured that the exercise data acquisition and analysis module can be connected to the personalized modeling and recommendation system module. This helps to use the analysis result for personalized modeling and provide the patient with a more accurate exercise plan.

By means of the above implementation, the exercise data acquisition and analysis module can efficiently obtain the patient's exercise data from the exercise tracking devices or applications and achieve real-time performance and accuracy by using an advanced data analysis algorithm. Module connection ensures cooperative operation with other key modules, thereby providing the patient with more comprehensive exercise information and personalized services. Specifically, the multi-channel integration module is implemented as follows:

Unified API: A unified API is designed, so that a core function of the chatbot can be invoked by means of the interface. This ensures consistency and compatibility on different platforms.

Responsive design: A responsive design principle is used, so that an interface of the chatbot can adapt to screen sizes and resolution of different terminals. This helps to provide a good user experience on a mobile application, a web page, and other different platforms.

Mobile application development: Mobile application platforms (IOS, Android, and the like) are developed, and hardware functions of a mobile device, such as a sensor and a push notification, are full used. Performance of mobile applications and user experience are ensured.

Web-side implementation: A Web-based user interface is created, so that the user can access the chatbot via a browser. Compatibility on different browsers is ensured, and a good Web-side experience is provided.

API security: A secure API authentication and authorization mechanism is used to ensure secure and reliable data transmission on different platforms. Hypertext transfer protocol secure (HTTPS) and other security protocols are used to encrypt data transmission.

Adaptive design: An adaptive design is achieved, so that the chatbot automatically can adjust a layout and functions on different platforms based on a user device and environment. This helps to provide a more flexible user experience.

Connection to the user information and health record module: By means of a clearly-defined API, it is ensured that the multi-channel integration module can be connected to the user information and health record module. This helps to maintain consistency of user information on different platforms.

Connection to the personalized modeling and recommendation system module: By means of a clearly-defined API, it is ensured that the multi-channel integration module can be connected to the personalized modeling and recommendation system module. This helps to provide consistent personalized services on different platforms.

By means of the above implementation, the multi-channel integration module enables the chatbot to run on a mobile application, a web page and other different platforms, thereby maintaining consistency and compatibility, and providing the patient with more flexible selection and use experience.

Specifically, the social interaction module is implemented as follows:

User registration and personal data: A user registration function is provided, so that the patient can create a personal account and fill in personal data. The information may include the user's interest, exercise preference, health goal, and the like.

User search and matching: A user search and matching function is achieved, so that the patient can find and connect to other users with similar interest or the same health goal. This can be matched based on the user's health records and exercise habits through the support of the personalized modeling and recommendation system module.

Real time chat and discussion section: A real-time chat function is provided, so that patients can interact directly. A discussion section is created, so that the user can share experiences, raise questions, and exchange suggestions. The understanding of a natural language is achieved by using the NLP module, making the interaction more intelligent and natural.

User dynamics and achievement display: Patients can share their exercise achievements and progress. This may be a number of steps per day, an exercise goal achieved, health improvement, and the like. Personalized rewards and encouragement can be provided by the personalized modeling and recommendation system module.

Group creation and management: The user is allowed to create an exercise group for a wider range of social interaction. Group management functions, including member management, group goal setting, and the like, are provided.

Push notification and reminder: The user can know group activities, new chat information, and the like in time by using a push notification function. This helps to promote activity of social interaction.

User feedback and suggestions: A channel is provided for the user to provide a feedback and suggestions to continuously optimize functions of the social interaction module. This helps to meet user needs and improve user satisfaction.

Connection to the personalized modeling and recommendation system module: By means of a clearly-defined API, it is ensured that the social interaction module can be connected to the personalized modeling and recommendation system module. This helps to adjust personalized modeling based on the user's social behavior and provide more targeted social interaction suggestions.

By means of the above implementation, the social interaction module can provide an interactive platform for patients to share experiences and build communities, and provide a deeper and more personalized social experience with the support of the personalized modeling and recommendation system.

Specifically, the security and privacy module is implemented as follows:

Data encryption: The patient's health data is encrypted to ensure security during storage and transmission. A powerful encryption algorithm, such as an advanced encryption standard (AES), is used to prevent unauthorized access.

Access control: A strict access control mechanism is implemented to ensure that only authorized personnel can access the patient's health data. This includes authentication, permission management, and the like, to restrict access to sensitive information.

Anonymization: If possible, the health data is anonymized to reduce an identification risk of the individual patient. This helps to balance a relationship between data utilization and privacy protection.

Compliance and standards: The security and privacy module follows relevant privacy laws and standards, such as American Health Insurance Portability and Accountability Act (HIPAA) and European General Data Protection Regulation (GDPR), to ensure the compliance of the system. The system is updated in time to adapt to new regulatory requirements.

Security audit and monitoring: A security audit and monitoring mechanism is implemented to track a security event and an access record in the chatbot. This helps to find potential security threats in time and take appropriate measures.

Security training: Security training is provided to a system operator to enhance the operator's awareness of privacy and security. This includes how to deal with sensitive information and avoid social engineering attacks, and the like.

User permission management: Fine-grained user permission management is implemented in the chatbot to ensure that each user can only access data and functions required by the user's job responsibilities. This helps to reduce potential internal risks.

Security update and vulnerability management: Regular security update is performed on the system, and known vulnerabilities are fixed in time. A vulnerability management mechanism is implemented, and security assessment and penetration testing are regularly performed.

Connection to other modules: By means of a clearly-defined API, it is ensured that the security and privacy module can be connected to the user information and health record module. This helps to ensure the comprehensive protection of security and privacy in the whole system.

By means of the above implementation, the security and privacy module can fully ensure the patient's health data security and ensure that privacy protection and security are fully considered during design and operation of the system.

Finally, it should be noted that the above descriptions are only preferred embodiments of the present disclosure, and are not intended to limit the present disclosure. Although the present disclosure has been described in detail with reference to the foregoing embodiments, those skilled in the art may still modify the technical solutions described in the foregoing embodiments, or equivalently substitute some technical features thereof. Any modification, equivalent substitution and improvement made within the spirit and principle of the present disclosure should fall within the protection scope of the claims of the present disclosure.

1. A chatbot for promoting exercise habit formation of a patient with chronic diseases, comprising:
a processor; and a memory having program instructions stored thereon,
wherein when the processor executes the program instructions stored on the memory, the processor is configured to performing following operations by using following modules:
a user information and health record module configured to store a patient's personal information, a health status and medical history by using a database and connected to a personalized modeling and recommendation system module;
the personalized modeling and recommendation system module configured to establish a personalized exercise plan recommendation system by machine learning algorithms based on user information and a health record and connected to the user information and health record module and a neuro linguistic programming (NLP) module;
the NLP module configured to process a natural language input of a user and provide a dialogue and connected to the personalized modeling and recommendation system module, the user information and health record module, and a real-time feedback system module, wherein the NLP module is implemented by using speech recognition technology, text analysis and understanding, emotional analysis, entity recognition, dialogue management, intention recognition and multilingual support;
the real-time feedback system module configured to provide a real-time exercise feedback, comprising exercise data and suggestions, and connected to the personalized modeling and recommendation system module, an exercise data acquisition and analysis module, and the NLP module;
the exercise data acquisition and analysis module configured to obtain the patient's exercise data from an exercise tracking device or application for analysis and interpretation and connected to the real-time feedback system module and the personalized modeling and recommendation system module;
a multi-channel integration module configured to enable the chatbot to run on different platforms and connected to the personalized modeling and recommendation system module, the user information and health record module, and the NLP module;
wherein the multi-channel integration module is configured to:
design an application programming interface (API), so that a function of the chatbot is invoked by means of the interface;
use a responsive design principle, so that an interface of the chatbot adapts to screen sizes and resolution of different terminals;
develop a mobile application platform and use hardware functions of a mobile device;
create a Web-based user interface, so that the user accesses the chatbot via a browser;
use an authentication and authorization mechanism of the API to ensure secure and reliable data transmission on different platforms;
achieve adaptive design, so that the chatbot automatically adjusts a layout and functions on different platforms based on a user device and environment; and
ensure that the multi-channel integration module is connected to the user information and health record module and the personalized modeling and recommendation system module;
a social interaction module configured to provide a social interaction function and connected to the personalized modeling and recommendation system module and the NLP module;
wherein the social interaction module is configured to:
provide a user registration function to allow the patient to create a personal account and fill in personal data;
achieve a user search and matching function, so that the patient finds and connects to other users with similar interest or the same health goal;
provide a real-time chat function, so that patients interact directly;
allow the patient to share exercise achievements and progress;
allow the user to create an exercise group for a wider range of social interaction;
allow the user to know group activities and new chat information in time by using a push notification function;
provide a channel for the user to provide a feedback and suggestions to continuously optimize functions of the social interaction module; and
ensure, by means of the API, that the social interaction module is connected to the personalized modeling and recommendation system module;
a security and privacy module configured to ensure that the patient's health data is securely stored and processed, and connected to the user information and health record module;
wherein the security and privacy module is configured to:
encrypt the patient's health data to ensure security during storage and transmission;
implement an access control mechanism to ensure that only authorized personnel access the patient's health data;
anonymize the health data to reduce an identification risk of the individual patient;
implement a security audit and monitoring mechanism to track a security event and an access record in the chatbot;
implement user permission management in the chatbot to ensure that each user only accesses data and functions required by the user's job responsibilities;
regularly update security of the chatbot, fix vulnerabilities in time, implement a vulnerability management mechanism, and regularly perform security assessment and penetration testing; and
ensure, by means of the API, that the security and privacy module is connected to the user information and health record module.

2. The chatbot for promoting exercise habit formation of a patient with chronic diseases according to claim 1, wherein the user information and health record module is configured to:
use the database to store the patient's personal information and health record, comprising name, age, gender, physical condition, past medical history, and medication;
implement data encryption for storage of personal health information;
introduce a user authentication mechanism to ensure that only legally authorized personnel access and modify the patient's information;
provide the API so that other modules access the patient's information;
update the database when the patient updates the personal information or has a new medical record; and
provide a user interface to enable the patient to view and edit the personal information.

3. The chatbot for promoting exercise habit formation of a patient with chronic diseases according to claim 2, wherein the personalized modeling and recommendation system module is configured to:
establish a personalized model by using a data analysis technology based on the patient's personal information and health record data, to identify the patient's exercise preference, physical condition and response mode;
develop a rule engine to make an exercise plan for the patient based on medical knowledge and health guidelines;
adjust the exercise plan when the patient's health status changes or a new medical record is added;
introduce a feedback loop to continuously adjust the model by monitoring the patient's exercise feedback and progress;
ensure, by means of the API, that the personalized modeling and recommendation system module accesses and updates data of the user information and health record module; and
enable, by the NLP module, the personalized exercise plan recommendation system to understand a natural language input of the patient, and adjust an output of the personalized model based on the patient's questions and needs.

4. The chatbot for promoting exercise habit formation of a patient with chronic diseases according to claim 3, wherein the NLP module is configured to:
convert the user's oral input into text;
use a natural language processing technology to analyze and understand the user's text input to extract a user intention and key information;
consider integrating emotional analysis to understand the user's emotional state;
extract a key entity in the user input by using an entity recognition technology;
introduce dialogue management to keep a context of the dialogue and ensure coherence and fluency;
determine the user's specific intention through intention recognition; and
ensure, by means of the API, that the NLP module is connected to the personalized modeling and recommendation system module, the user information and health record module, and the real-time feedback system module.

5. The chatbot for promoting exercise habit formation of a patient with chronic diseases according to claim 4, wherein the real-time feedback system module is configured to:
connect to the exercise data acquisition and analysis module to receive the real-time exercise data;
perform real-time processing and analysis on the received exercise data; and use an algorithm comprising a real-time data stream processing technology;
generate a personalized exercise feedback based on an analysis result;
ensure real-time performance of feedback information, to enable the patient to know the patient's own exercise status and obtain suggestions in time, and achieve personalized adjustment of a real-time feedback with the personalized modeling and recommendation system module;
connect to the NLP module, receive the user's natural language input, and adjust feedback content in time; and
ensure, by means of the API, that the real-time feedback system module is connected to the personalized modeling and recommendation system module.

6. The chatbot for promoting exercise habit formation of a patient with chronic diseases according to claim 5, wherein the exercise data acquisition and analysis module is configured to:
access the exercise tracking device or application;
communicate with the exercise tracking device or application by means of the API to obtain the real-time exercise data;
clean and preprocess collected original data to deal with an abnormal value and a missing value;
use an efficient data acquisition algorithm to ensure real-time performance of the data;
use a data analysis algorithm to analyze the exercise data;
use a caching and streaming technology to achieve real-time processing of a large data volume;
use a machine learning technology to analyze the exercise data, comprising exercise pattern recognition, exercise habit analysis, and exercise effect evaluation, to obtain deeper exercise information;
generate a readable analysis report based on an analysis result, comprising the patient's exercise trend and health status evaluation; and
ensure, by means of the API, that the exercise data acquisition and analysis module is connected to the real-time feedback system module and the personalized modeling and recommendation system module.

7-9. (canceled)
